(19) United States
(12) Patent Application Publication
(10) Pub. No.: US 2008/0097309 A1
(43) Pub. Date: Apr. 24, 2008

(54) PROTECTIVE SLEEVE FOR MEDICAL DEVICE COMPONENTS

(75) Inventors: BRADLEY J. ENEGREN, MOORPARK, CA (US); POONAM S. GULATI, LA CANADA, CA (US)

Correspondence Address: MEDTRONIC MINIMED INC., 18000 DEVONSHIRE STREET, NORTHRIDGE, CA, 91325-1219, US

(73) Assignee: MEDTRONIC MINIMED, INC., NORTHRIDGE, CA (US)

(21) Appl. No.: 11/463,258

(22) Filed: Aug. 8, 2006

(51) Int. Cl. A61M 5/142

(52) U.S. Cl. 604/111; 604/131

(57) ABSTRACT

An external infusion device comprising a housing, a motor contained within the housing, programmable electronics, a fluid reservoir, and a reservoir sleeve, where the fluid reservoir is encapsulated by the reservoir sleeve. The reservoir sleeve may be made of rubber, neoprene, or the like, and may be supported by a metal mesh-like frame support structure that is infused within the material. Additionally, the fluid reservoir may be made of plastic or glass, and contain insulin or other fluids for administering to humans.

### FIELD OF THE INVENTION

Embodiments of the invention relate to improvements in the durability and functionality of fluid reservoirs in external infusion devices. Specifically, embodiments of the invention teach a method for providing protection to fluid reservoirs during the packaging, transport, and/or usage phases.

### BACKGROUND OF THE INVENTION

Infusion devices and systems have become relatively prevalent in the medical field for use in delivering or dispensing prescribed medication such as insulin to a patient. In one form, such devices comprise a transportable, pocket-size pump housing capable of receiving a reservoir of medication for administration to the patient through an associated catheter or infusion set.

Infusion devices have significant advantages over traditional medication delivery methods because of their precision, consistency, and versatility. Patients are able to set exact dosage amounts and generally benefit from receiving medication from pumps during physical activity or other occasions that would otherwise not easily be suited for doing so. As a result, infusion pumps have effectively reduced the restrictions that a diabetic patient's medical needs place upon him allowing the patient to live a more active and fulfilling lifestyle.

### SUMMARY OF THE DISCLOSURE

According to an embodiment of the invention, an external infusion device includes a housing, a motor contained within the housing, programmable electronics, a fluid reservoir, and a reservoir sleeve. In particular embodiments, the fluid reservoir is encapsulated by the reservoir sleeve. In some embodiments, the fluid reservoir contains insulin. In other embodiments, the reservoir sleeve is made of rubber and/or neoprene. In still additional embodiments, the reservoir sleeve includes a metal or metal alloy mesh-like frame support structure. In still further embodiments, the reservoir sleeve is adapted to encapsulate an insulin reservoir made of glass, plastic and/or Topas®. In additional embodiments, the reservoir sleeve is responsive to a change in temperature, and, in particular, changes color in response to a change in temperature. In other embodiments, the reservoir sleeve is responsive to a change in moisture, and, in particular, changes color in response to the change in moisture. In still further embodiments, the reservoir sleeve is comprised of a leak-proof material. In additional embodiments, the thickness of the reservoir sleeve is adapted in size to fit a variety of fluid reservoir sizes.

In other embodiments, a reservoir containing fluid includes a base, a cylindrical body, an opening at one end of the body to allow for fluid transfer, and a sleeve for the reservoir. In some embodiments, the sleeve fits securely around the reservoir and, in other embodiments, the sleeve for the reservoir is adapted for safely storing and transporting the reservoir without breakage. In additional embodiments, the sleeve for the reservoir is adapted for use with an external infusion device. In some embodiments, the reservoir contains insulin. In further embodiments, the sleeve for the reservoir is made of rubber and/or neoprene. In still additional embodiments, the reservoir is comprised of a material that contains a metal or metal alloy mesh-like frame support structure. In some embodiments, the sleeve for the reservoir is adapted to encapsulate a reservoir made of glass, plastic and/or Topas®. In other particular embodiments, the sleeve for the reservoir is responsive to a change in temperature and, in particular, the reservoir changes color in response to a change in temperature. In other embodiments, the sleeve for the reservoir is responsive to the change in moisture and, in particular, the sleeve for the reservoir changes color in response to the change in moisture. In additional embodiments, the sleeve for the reservoir is comprised of a leak-proof material and/or the thickness of the sleeve for the reservoir is adapted in size to fit a variety of fluid reservoir sizes.

Other features and advantages of the invention will become apparent from the following detailed description, taken in conjunction with the accompanying drawings which illustrate, by way of example, various features of embodiments of the invention.

### BRIEF DESCRIPTION OF THE DRAWINGS

A detailed description of embodiments of the invention will be made with reference to the accompanying drawings, where like numerals designate corresponding parts or cross-sections in the several figures.

FIG. 1 shows a perspective view of an infusion device in accordance with an embodiment of the present invention.

FIG. 2 depicts a cross-sectional view of a reservoir sleeve according to a particular embodiment of the present invention.

FIG. 3 is a side view of a reservoir sleeve according to a particular embodiment of the present invention.

FIG. 4 shows a perspective view of a reservoir sleeve according to a different embodiment of the present invention.

### DETAILED DESCRIPTION OF THE PREFERRED EMBODIMENTS

As shown in the drawings for purposes of illustration, the invention is embodied in a protective sleeve to provide insulation, durability, and impact resistance during the packaging, transport and/or usage phases of a reservoir, catheter set and/or any other external, easily portable attachment developed to deliver fluid to humans. In particular embodiments, the protective sleeve may be used with an insulin reservoir for an external infusion device of the type described in U.S. Pat. No. 5,554,798 entitled “External Infusion Device with Remote Programming, Bolus Estimator and/or Vibration Alarm Capabilities,” which is specifically incorporated by reference herein. In additional embodiments, the infusion device may include an in-line drive system of the type described in U.S. Pat. No. 6,248,093 entitled “Compact Pump Drive System,” which is specifically incorporated by reference herein. In further embodiments, the infusion device may be a patch pump, or any other type of external pump that facilitates the delivery of fluid into the body. In alternative embodiments, the sleeve may be used to protect other devices or attachments that are used to contain, transfer and/or administer fluids such as medication, drugs, vitamins, vaccines, hormones, antigens, water or the like. Such alternative embodiments may be adapted either for mobile or stationary fluid delivery devices.

An external infusion device typically includes a housing to enclose the pump drive system, a fluid containment assembly and a power supply. The device's drive system generally includes a small motor (DC, stepper, solenoid or other type) and drive train components such as gears, screws and levers that act in concert to convert rotational motor motion to translational displacement of a stopper in the fluid reservoir. In some embodiments, the drive system may use gas, nitric or other types of pressure systems that induce displacement of a stopper in the fluid reservoir. The fluid containment assembly generally includes the reservoir with a stopper, flexible tubing and a catheter or infusion set that transports the fluid or medication from the infusion device to the body of the user. The device's electronic system may include programmable controls for regulating the motor, as well as for setting desired dosage intervals over a certain period of time. In other embodiments, the external device may be a patch pump, or any other type of external pump that delivers fluid to the user.

In general, subjecting a fluid reservoir to a variety of physical and/or thermal conditions could make it more susceptible to cracking or breakage. Providing cushioning across the reservoir's surface area and/or at identifiable stress points would serve to mitigate damage to the reservoir and decrease the likelihood of fluid leakage.

Moreover, in some instances, it may be important to stabilize the temperature of fluids within a reservoir during transportation, storage or usage. In particular embodiments, the protective sleeve may be a reservoir sleeve including a material that could aid in maintaining a particular fluid temperature within the reservoir. Sleeves may be made from different fluid insulation materials including, but not limited to foam, neoprene, rubber or the like. Stabilization of fluid temperature may give the pump user wider discretion in his or her choice of participating in warm and/or cold weather activities (i.e. desert hiking, snow skiing, or the like). In particular embodiments, the reservoir sleeve may be used with disposable reservoirs, pre-filled reservoirs, patient filled reservoirs or the like.

In other embodiments, the protective sleeve may form a protective covering for refillable cartridges, containers or the like. In still additional embodiments, fluid reservoirs or containers may be made from different materials including, but not limited to glass, ceramic, plastic, or the like. Further embodiments may be used with Topas® and/or COC reservoirs of the type described in U.S. patent application Ser. No. 11/100,188 filed on Apr. 5, 2005 and entitled “Improved Fluid Reservoir for Use with An External Infusion Device,” which is specifically incorporated by reference herein.

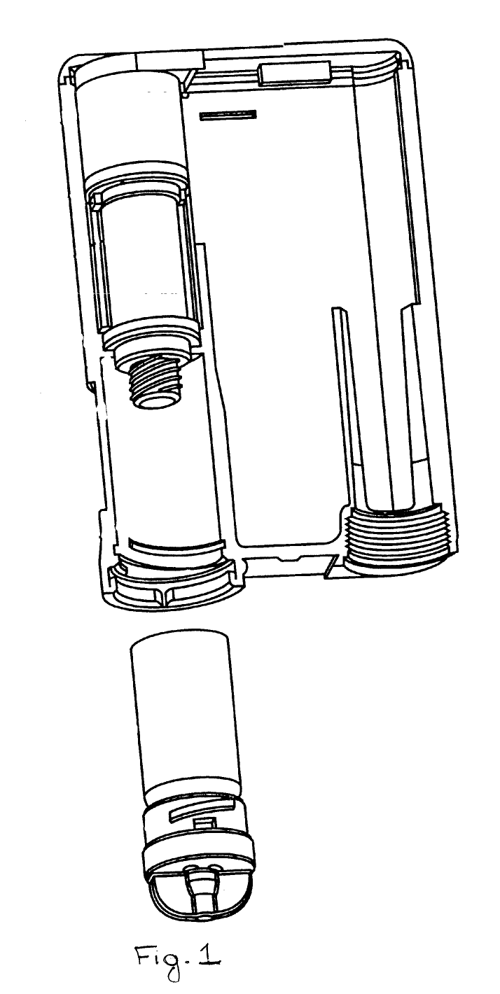

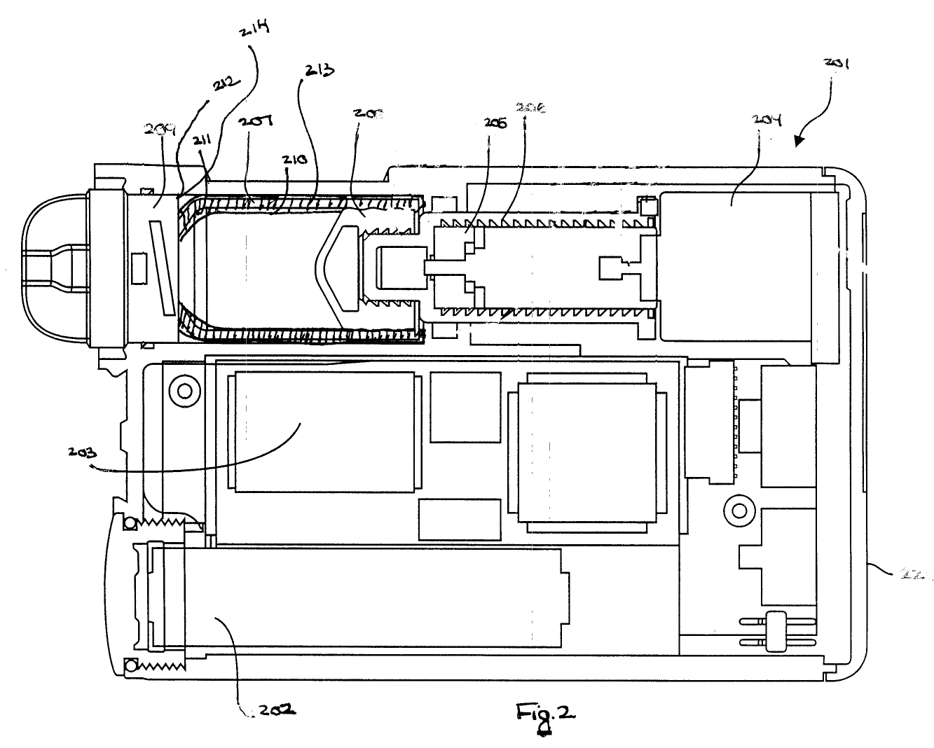

FIG. 1 shows an embodiment of an infusion device and its associated drive system. In particular embodiments, as shown in FIG. 2, an infusion pump 201 includes a power supply 202, electronic control circuitry 203, a drive mechanism such as a motor 204 (i.e., solenoid, stepper, or DC), a first drive member, such as an externally threaded drive gear or screw 205, a second drive member such as an internally threaded plunger gear or slide 206, and a removable reservoir 207. Such components are contained within a housing 220. The reservoir 207 includes a plunger or piston assembly 208 with O-rings or integral raised edges for forming a water and airtight seal. The reservoir 207 is secured to the housing 220 with a connector 209 that serves as the interface between the reservoir 207 and the infusion set tubing (not shown). The fluid containing reservoir 207 includes reservoir barrel 210, a neck 211, and a head 212 which are generally concentrically aligned. The reservoir sleeve 213 fits tightly around the reservoir barrel 210, neck 211, and head 212. The reservoir sleeve 213 may have different thickness measurements to vary its protective and/or insulating properties. The reservoir sleeve 213 may further aid to cushion the reservoir 207 within pump 201 during its storage, transport and/or usage phases. The neck 211, which has a smaller diameter then the barrel 210, connects a front end of the barrel 210 to the head 212. The neck 212 sits within an outlet port 214 formed in the housing 220. The head 212, which has a larger diameter than the neck 211, extends through the housing 220 and into the connector 209, which establishes fluid communication from the barrel 210, through the housing 220 and into the infusion set tubing (not shown).

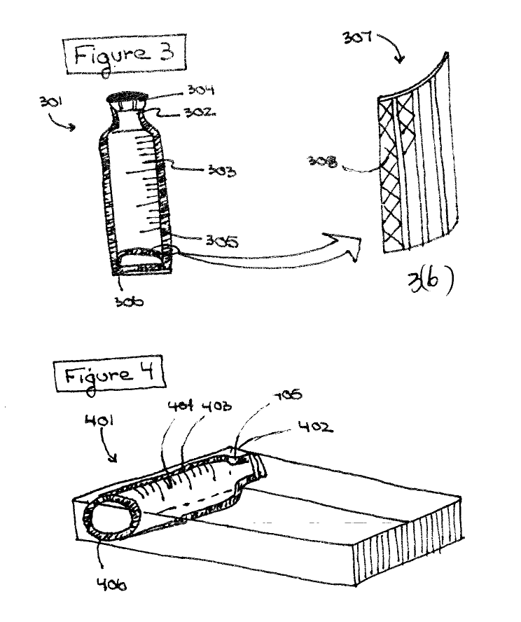

FIG. 3 depicts a particular embodiment of the invention where a fluid reservoir 301 is protected by the reservoir sleeve 305, which forms a snug sleeve-like fit around the reservoir barrel 303, base perimeter 306 and the neck 302 up to the head 304. Such a sleeve may have circumferential lengths across the reservoir neck and base that range from 0.01 to 1 inch to allow for appropriate protection of the encapsulated fluid. Other embodiments may include circumferential lengths larger than 1 inch or smaller than 0.01 inches at each facial surface. A cross section 307 of the reservoir sleeve depicts a metal or alloy frame-like structure 308, which may be used within the sleeve material to give added shape and/or fit. The frame-like structure 308 may either be attached to the outside of the sleeve or infused within its material. Such a structure may further mitigate risk to the reservoir user by keeping the reservoir barrel 303 intact should it shatter into separate pieces. Additionally, in other embodiments, the reservoir sleeve 305 may be made from leak-proof material to prevent the fluid reservoir 301 from leaking should the reservoir barrel 303 or neck 302 crack or shatter. The fluid reservoir 301 may be used within an external infusion device or within any other mobile or stationary fluid dispensing device. The fitting above the head 301 serves to transport fluid via tubing, funnel, dispenser or other method of transfer.

FIG. 4 shows another particular embodiment of the invention where the fluid reservoir 401 is encapsulated by the reservoir sleeve 403 and located inside a carton 402 for transport. The reservoir sleeve 403 may have adhesive like qualities to ensure minimal movement within the carton and to aid in keeping the fluid reservoirs from colliding. Moreover, an increased thickness of the protective material may further act to decrease the chance for breakage caused by collisions or other forms of impact.

While the description above refers to particular embodiments of the present invention, it will be understood that many modifications may be made without departing from the spirit thereof. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

What is claimed is:

1. An external infusion device comprising:
a housing;
a motor contained within the housing;
programmable electronics;
a fluid reservoir; and
a reservoir sleeve,
wherein the fluid reservoir is encapsulated by the reservoir sleeve.

2. An external infusion device according to claim 1, wherein the fluid reservoir contains insulin.

3. An external infusion device according to claim 1, wherein the reservoir sleeve is made of rubber.

4. An external infusion device according to claim 1, wherein the reservoir sleeve is made of neoprene.

5. An external infusion device according to claim 1, wherein the reservoir sleeve further includes a metal or metal alloy mesh-like frame support structure.

6. An external infusion device according to claim 1, wherein the reservoir sleeve is adapted to encapsulate an insulin reservoir made of glass.

7. An external infusion device according to claim 1, wherein the reservoir sleeve is adapted to encapsulate an insulin reservoir made of plastic.

8. An external infusion device according to claim 1, wherein the reservoir sleeve is adapted to encapsulate an insulin reservoir made of Topas®.

9. An external infusion device according to claim 1, wherein the reservoir sleeve is responsive to a change in temperature.

10. An external infusion device according to claim 9, wherein the reservoir sleeve changes color in response to a change in temperature.

11. An external infusion device according to claim 1, wherein the reservoir sleeve is responsive to a change in moisture.

12. An external infusion device according to claim 11, wherein the reservoir sleeve changes color in response to the change in moisture.

13. An external infusion device according to claim 1, wherein the reservoir sleeve is comprised of a leak-proof material.

14. An external infusion device according to claim 1, wherein the thickness of the reservoir sleeve is adapted in size to fit a variety of fluid reservoir sizes.

15. A reservoir containing fluid, the reservoir comprising:
a base;
a cylindrical body;
an opening at one end of the body to allow for fluid transfer; and
a sleeve for the reservoir,
wherein the sleeve fits securely around the reservoir.

16. A reservoir according to claim 15, wherein the sleeve for the reservoir is adapted for safely storing and transporting the reservoir without breakage.

17. A reservoir according to claim 15, wherein the sleeve for the reservoir is adapted for use with an external infusion device.

18. A reservoir according to claim 15, wherein the reservoir contains insulin.

19. A reservoir according to claim 15, wherein the sleeve for the reservoir is made of rubber.

20. A reservoir according to claim 15, wherein the sleeve for the reservoir is made of neoprene.

21. A reservoir according to claim 15, wherein the sleeve for the reservoir is comprised of a material that contains a metal or metal alloy mesh-like frame support structure.

22. A reservoir according to claim 15, wherein the sleeve for the reservoir is adapted to encapsulate a reservoir made of glass.

23. A reservoir according to claim 15, wherein the sleeve for the reservoir is adapted to encapsulate a reservoir made of plastic.

24. A reservoir according to claim 15, wherein the sleeve for the reservoir is adapted to encapsulate a reservoir made of Topas®.

25. A reservoir according to claim 15, wherein the sleeve for the reservoir is responsive to a change in temperature.

26. A reservoir according to claim 15, wherein the sleeve for the reservoir changes color in response to a change in temperature.

27. A reservoir according to claim 15, wherein the sleeve for the reservoir is responsive to the change in moisture.

28. A reservoir according to claim 15, wherein the sleeve for the reservoir changes color in response to the change in moisture.

29. A reservoir according to claim 15, wherein the sleeve for the reservoir is comprised of a leak-proof material.

30. A reservoir according to claim 15, wherein the thickness of the sleeve for the reservoir is adapted in size to fit a variety of fluid reservoir sizes.
